(19) United States
(12) Patent Application Publication
(10) Pub. No.: US 2017/0307452 A1
(43) Pub. Date: Oct. 26, 2017

(54) Tensiometer

(71) Applicant: Jesse Lafian, Athens, GA (US)

(72) Inventor: Jesse Lafian, Athens, GA (US)

(21) Appl. No.: 15/495,961

(22) Filed: Apr. 24, 2017

Related U.S. Application Data
(60) Provisional application No. 62/326,410, filed on Apr. 22, 2016.

(51) Int. Cl. G01L 1/00; G01N 27/00; G01N 33/24; G01N 3/08

(52) U.S. Cl. CPC G01L 1/005; G01N 33/246; G01N 3/08; G01N 27/00; G01N 2033/245

(57) ABSTRACT

Tensiometer device for measuring soil water tension. A pair of screws secures a load cell or strain gauge to an inner frame, a dowel pin transmits force to the load cell, a polymer chamber is enclosed on one side by a rubber dam that retains the polymer within the polymer chamber, and a hydrophilic porous window covers the rubber dam. A second pair of screws secure an outer frame to the inner frame holding the components of one or more tensiometers spaced across the frame, and an end cap. The load cell acts as a strain gauge transferring the force exerted on it as a change in electrical voltage that can be converted to a soil water tension (SWT) measurement.

### CROSS REFERENCE TO RELATED APPLICATIONS

This application claims priority from U.S. Patent Application Ser. 62/326,410, entitled “Tensiometer”, filed on 22 Apr. 2016. The benefit under 35 USC §119(e) of the United States provisional application is hereby claimed, and the aforementioned application is hereby incorporated herein by reference.

### FEDERALLY SPONSORED RESEARCH: Not Applicable

### SEQUENCE LISTING OR PROGRAM: Not Applicable

### TECHNICAL FIELD OF THE INVENTION

The present invention relates generally to tensiometers. More specifically, the present invention relates to a low cost, simple to assemble and use tensiometer device for measuring soil water tension (SWT) to help in water conservation efforts.

### BACKGROUND OF THE INVENTION

Water conservation is becoming increasingly important. As global temperatures reach record highs, severe drought limits the water supply to farms, cities, industries, and ecosystems. Over-irrigation can contribute to water shortages and suppress biodiversity by leaching nutrients that cause eutrophication. Improving irrigation accuracy could provide significant environmental and economic benefits worldwide.

One way to conduct irrigation is to schedule it based on the monitoring, management, and data of soil water tension (SWT). With the invention of tensiometers, SWT measurements have been used to determine optimal scheduled irrigation times. Precise irrigation scheduling based on SWT criteria is a powerful method to optimize plant performance. By using the ideal SWT and adjusting irrigation duration and amount, it is possible to simultaneously achieve high productivity and meet environmental stewardship goals for water use and reduced leaching nutrients that cause eutrophication.

There are different types of field instruments used to measure SWT, either directly or indirectly. What is needed is a low-cost tensiometer to measure soil water tension that can provider easier and cheaper measurement and adoption of schedule irrigation.

### Definitions

A barometer is a scientific instrument used in meteorology to measure atmospheric pressure. Pressure tendency can forecast short term changes in the weather. Numerous measurements of air pressure are used within surface weather analysis to help find surface troughs, high pressure systems and frontal boundaries.

A hydrogel is a network of polymer chains that are hydrophilic, sometimes found as a colloidal gel in which water is the dispersion medium. Hydrogels are highly absorbent (they can contain over 90% water) natural or synthetic polymeric networks. Hydrogels also possess a degree of flexibility very similar to natural tissue, due to their significant water content.

Microelectromechanical systems (or MEMS) barometers are extremely small devices between 1 and 100 micrometers in size (i.e. 0.001 to 0.1 mm). They are created via photolithography or photochemical machining. Typical applications include miniaturized weather stations, electronic barometers and altimeters.

Soil water tension (SWT) is the force necessary for plant roots to extract water from the soil.

A tensiometer is a device for measuring soil water tension.

### SUMMARY OF THE INVENTION

The device of the present invention measures soil water tension. That is how tightly water is held by the soil. Knowing this information is key to horticultural productivity, efficient water use, and water quality protection. Using this information allows growers and farmers to know more precisely when they should irrigate. In other words, it prevents over-watering.

A pair of screws secures a load cell or strain gauge to an inner frame, a dowel pin transmits force to the load cell, a polymer chamber is enclosed on one side by a rubber dam that retains the polymer within the polymer chamber, and a hydrophilic porous window covers the rubber dam. A second pair of screws secure an outer frame to the inner frame holding the components of one or more tensiometers spaced across the frame, and an end cap. The load cell acts as a strain gauge transferring the force exerted on it as a change in electrical voltage that can be converted to a soil water tension (SWT) measurement.

The present invention uses an enclosure for the polymer made up of a durable, hydrophilic, porous material whereas the prior art devices uses aluminum oxide ceramic. The materials cost is therefore reduced from approximately $2,400 to construct the prior art devices to approximately $50 to make the device of the present invention.

With fresh water becoming scarcer due to increasing demand from cities, industries, and interest groups, what is needed is a low-cost tensiometer in order to measure soil water tension (SWT)—the force necessary for plant roots to extract water from the soil.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 illustrates the assembled tensiometer of the present invention.

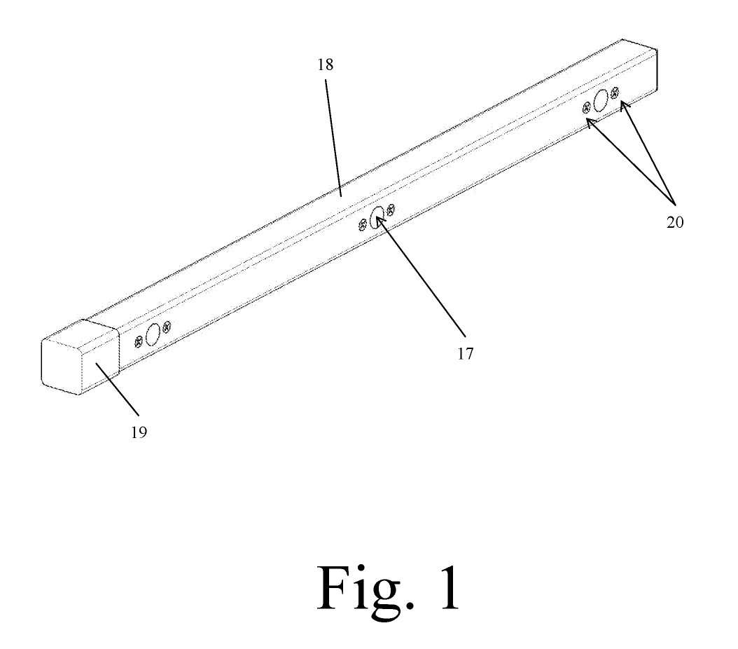

FIG. 2 illustrates an expanded view of the tensiometer frame of the present invention.

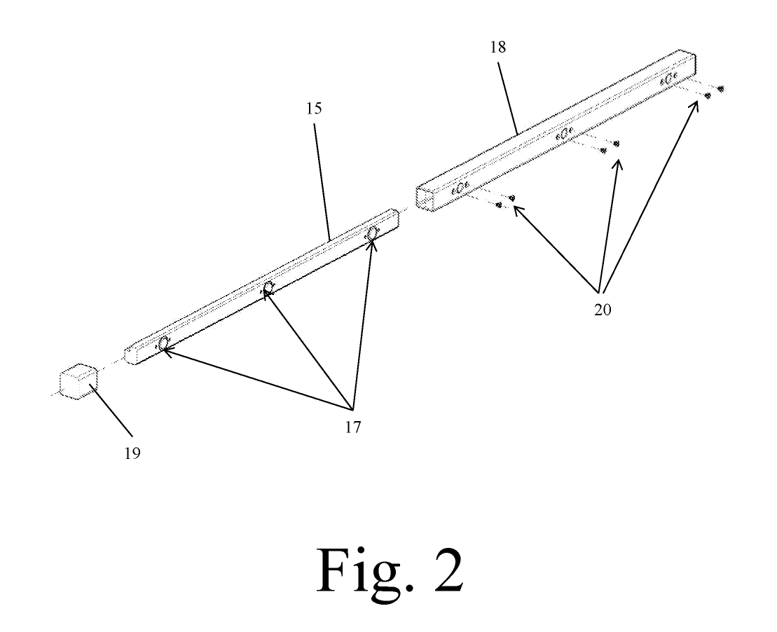

FIG. 3 illustrates an opposing expanded view of the tensiometer frame of the present invention where a strain gauge can be attached.

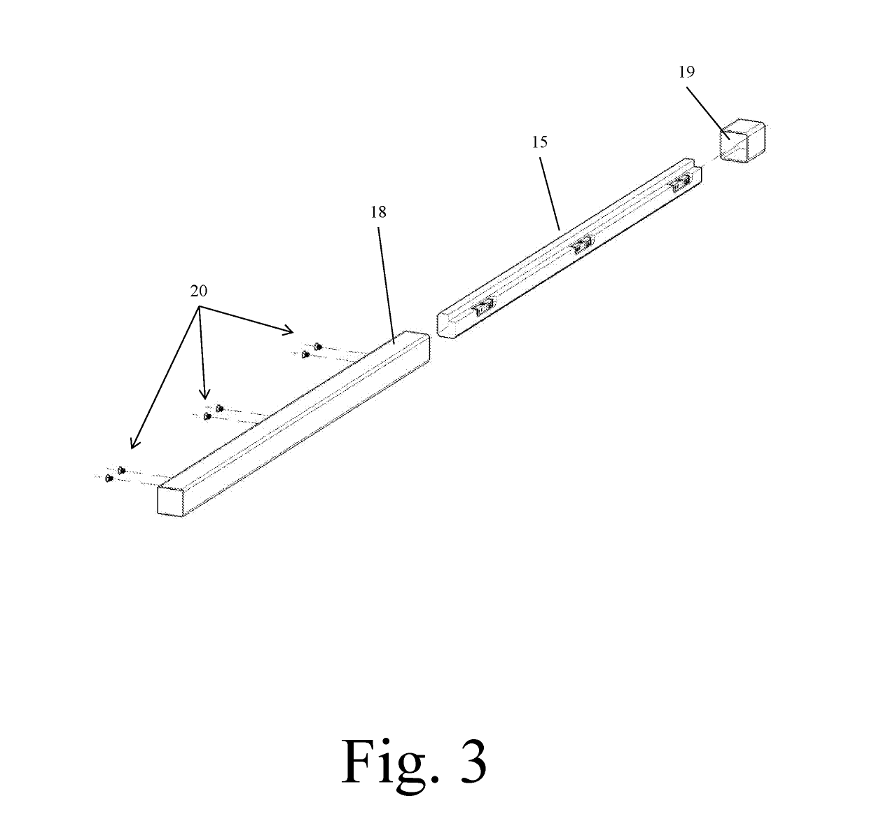

FIG. 4 illustrates an expanded view of the tensiometer frame of the present invention and how the strain gauge, dowel pin, rubber dam, and hydrophilic porous window are assembled.

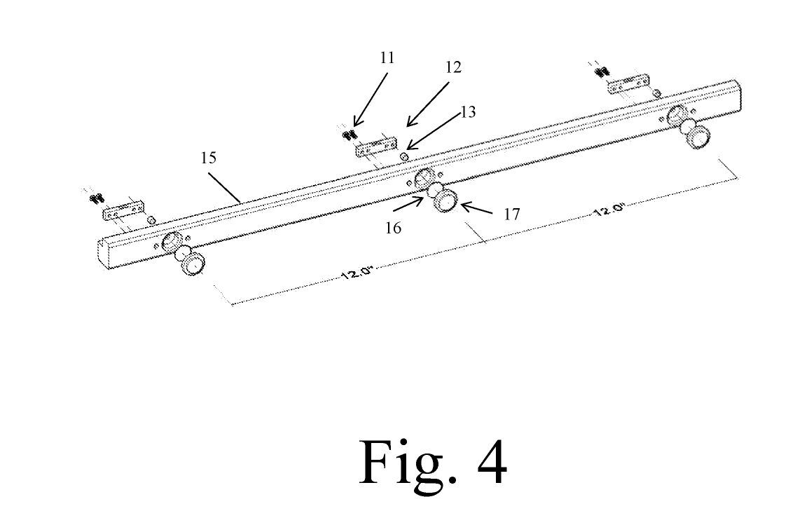

FIG. 5 illustrates an opposing expanded view of the tensiometer frame of the present invention and how the strain gauge, dowel pin, rubber dam, and hydrophilic porous window are assembled.

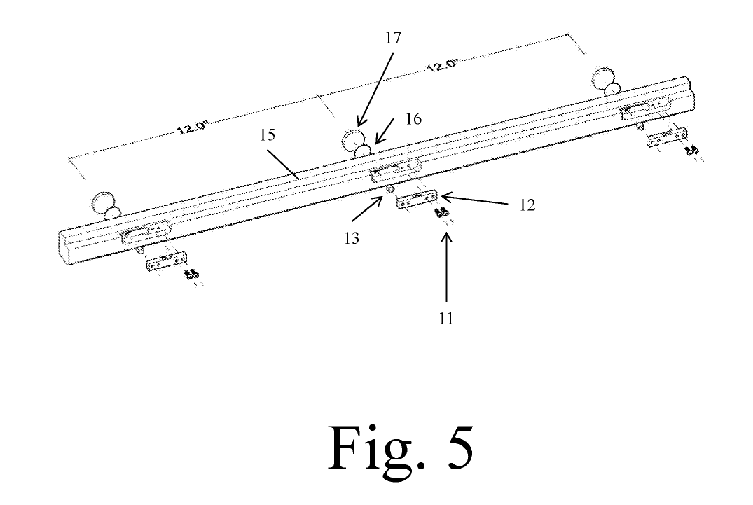

FIG. 6 illustrates an expanded, singular view of how the strain gauge, dowel pin, rubber dam, and hydrophilic porous window are assembled.

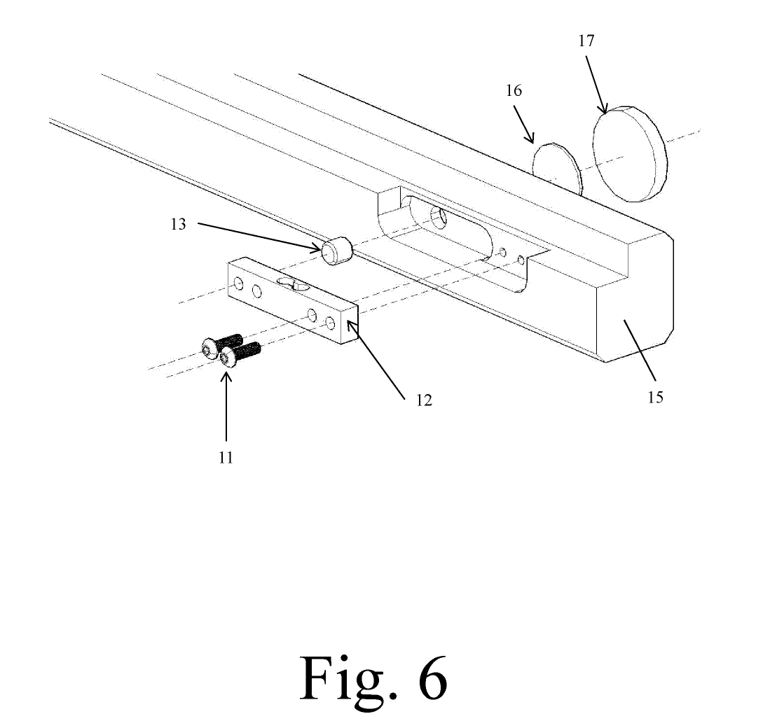

FIG. 7 is a top planar view of the complete assembly of the tensiometer of the present invention.

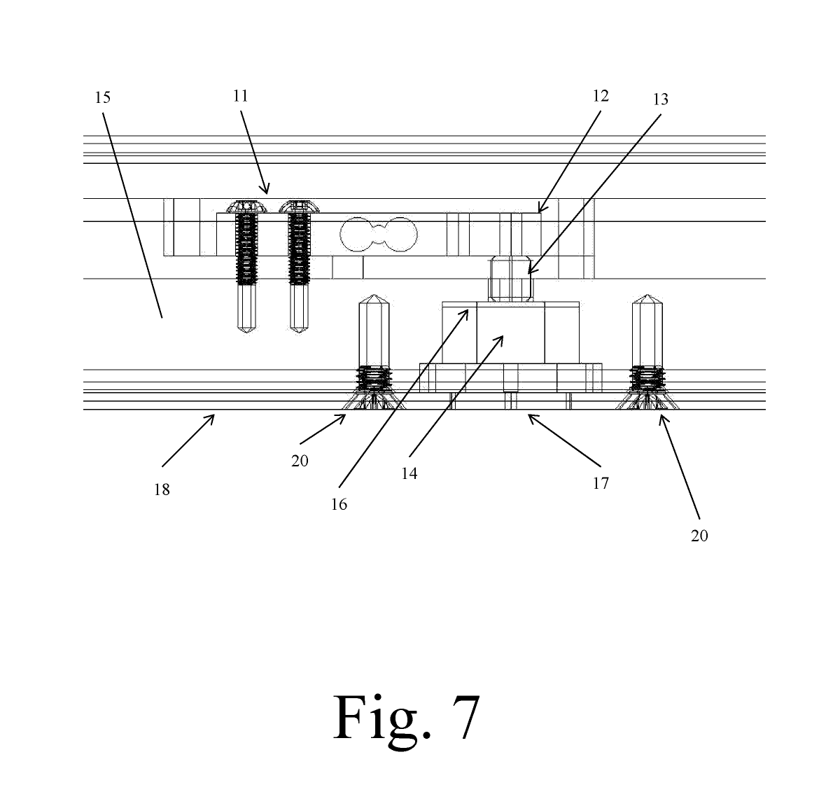

FIG. 8 is a perspective view of the complete assembly of the tensiometer of the present invention.

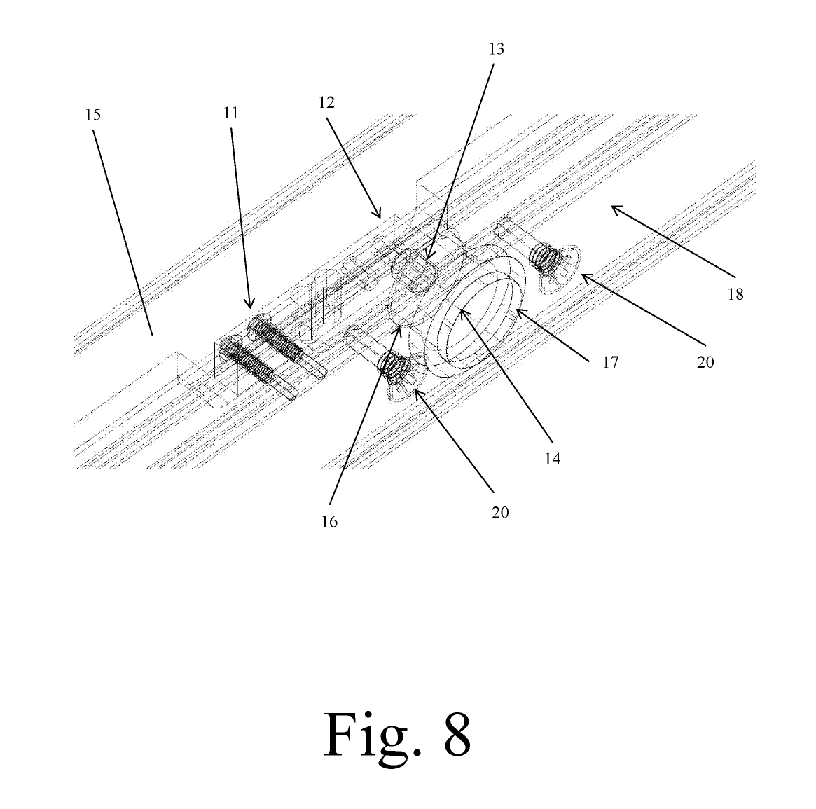

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

Water conservation is becoming increasingly important. As global temperatures reach record highs, severe drought limits water supply to farms, cities, industries, and ecosystems. Over-irrigation can contribute to water shortages and suppress biodiversity by leaching nutrients that cause eutrophication. Improving irrigation accuracy could provide significant environmental and economic benefits worldwide.

The objective of the present invention is to provide a low-cost tensiometer in order to measure soil water tension (SWT)—the force necessary for plant roots to extract water from the soil. As soil loses water, SWT increases. Irrigation becomes necessary when a plant's root zone reaches a certain degree of tension, which varies by plant type. Thus the ability to measure SWT is a key to improving irrigation accuracy.

The novel aspect of the present invention is that it mimics the response of plant roots to soil moisture; pressure accumulates in the device relative to the volume of water it extracts from the soil. This pressure signal is correlated with—and thus can be converted to—SWT.

Experimental results confirm that this tensiometer design could help improve irrigation accuracy; however, additional research is needed to increase the device's precision. When fully developed, the tensiometer could connect to an inexpensive automated irrigation control system for use in lawns, gardens, nurseries, greenhouses, and farms.

Researchers in various fields including agriculture, horticulture, ecology, geology, and hydrology could also benefit from this device. By optimizing the volume of water used for irrigation, this device may minimize the risk of overwatering, which can kill plants and therefore profits.

Finally, this tensiometer may drastically decrease water overconsumption and its associated costs. These include municipal, state, and federal expenses to ensure water quality and accessibility, as well as opportunity costs of ecosystem services such as fisheries production and CO2 sequestration.

Easily accessible soil water tension (SWT) data could enable growers to accurately determine how much water their plants need. During irrigation, SWT decreases to a point beyond which subsequent irrigation is unnecessary, costly, and wasteful.

As stated, the tensiometer of the present invention is a device for measuring soil water tension. As shown in the Figures, the tensiometer device of the present invention consists of a pair of screws 11 securing a load cell or strain gauge 12 to an inner frame 15, a dowel pin 13 transmits force to the load cell 12, a polymer chamber 14 is enclosed on one side by a rubber dam 16 that retains the polymer within the polymer chamber 14, a hydrophilic porous window 17 covers the rubber dam 16, a second pair of screws 20 secure an outer frame 18 to the inner frame 15 holding the components of one or more tensiometers spaced across the frame, and an end cap 19.

In addition to being prohibitively expensive and high-maintenance, many current water-filled tensiometers are unable to measure SWT beyond 0.8 bars, although some polymer osmotic tensiometers can measure up to 15 bar. In comparison to current tensiometers known in the prior art, the present invention does not require a membrane to prevent polymer leakage. This present invention uses fewer parts and a simpler design to require the change of failure and manufacturing costs.

In the present invention, the polymer is synthesized into macro-sized particles (>50 um) that cannot leak out through porous enclosure. This polymer material can consist of, but is not limited to, one of the following materials: polyethylene glycol, sodium polyacrylate, polyvinyl alcohol, polyvinyl pyrolidone, Cross-linked polyethylene glycol, Cross-linked sodium polyacrylate, Cross-linked polyvinyl alcohol, and Cross-linked polyvinyl pyrolidone.

In the prior art, a UGT tensiometer uses a membrane with extremely small pore sizes to contain the polymer. As a result, the UGT can only measure tension at one depth. In contrast, the present invention can measure tension at various and discrete depths in the soil profile.

The principle behind the new tensiometer device of the present invention is that polyacrylate can swell or shrink greatly, depending on how moist the environment is. When the polyacrylate is placed inside a chamber, with a strain gauge mounted to it via a dowel pin, it is possible to measure how strongly the polyacrylate is pushing against the strain gauge. That in turn depends on how much water the polyacrylate has absorbed and thus on how much water is available (i.e., plant available) in the soil. The pressure on the strain gauge can be measured with cheap microcontrollers and this information can be used to determine when a crop needs to be irrigated.

The hydrogel (sodium polyacrylate) inside the chamber expands to reach equilibrium with the water outside the chamber. That increases the pressure inside the chamber, which is transferred to a strain gauge via the dowel pin. The strain gauge sends a voltage to a data logger and computer. So, pressure buildup is measured via a change in voltage.

There are many different hydrogels that could work similarly. Sodium polyacrylate was chosen because of its easy access, availability, and affordability.

Pressure accumulates in the device relative to the volume of water it extracts from the soil. This pressure signal is correlated with, and thus can be converted to SWT. Experimental results confirm that this tensiometer design could help improve irrigation accuracy; however, additional research is needed to increase the device's precision.

The unexpected results and aspect of the present invention is that it mimics the response of plant roots to soil moisture. Pressure accumulates in the device relative to the volume of water it extracts from the soil. This pressure signal, which corresponds inversely to that of SWT, is then converted to a voltage. Because of its unique design, the tensiometer device of the present invention can measure a wider range of tension than present alternatives.

The present invention uses a load cell which measures force, where a UGT uses a pressure transducer which measures pressure. High-performance pressure transducers are expensive; they can cost $735. The Lafian Tensiometer uses a $6.00 load cell which is a benefit, and therefore a non-obvious improvement to the UGT tensiometer.

The present invention uses an enclosure for the polymer made up of a durable, hydrophilic, porous material; for example (but not limited to) one of the following materials: EASYCORK, EASYWOOD 3D ALGAE FILAMENT, BIOFILA, or WOODFILL whereas the UGT prior art device uses aluminum oxide ceramic. The materials cost is therefore reduced from approximately $2,400 retain to construct the prior art devices to approximately $50 to make the device of the present invention.

The present invention also costs less to make and is of a lower-maintenance construction. In an alternative embodiment, the tensiometer of the present invention could be constructed using a Microelectromechanical systems (or MEMS) barometer in place of the strain gauge and dowel pin. A MEMS barometer is an extremely small device between 1 and 100 micrometers in size (i.e. 0.001 to 0.1 mm).

Once fully developed, the tensiometer of the present invention could connect to an inexpensive automated irrigation control system for use in lawns, gardens, nurseries, greenhouses, farms, and research (e.g., in agriculture, horticulture, ecology, geology, and hydrology). By optimizing the volume of water used for irrigation, the tensiometer of the present invention may minimize the risk of overwatering, which can kill plants and therefore profits. Finally, the tensiometer of the present invention may reduce government expenditures on water quality/accessibility and prevent costs related to a lack of ecosystem services such as fisheries production and CO2 sequestration.

Thus, it is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. A tensiometer device comprising
an inner frame;
the inner frame having one or more tensiometers, each tensiometer comprising:
a polymer chamber;
the polymer chamber engaging a dowel pin on one side;
the polymer chamber covered by a rubber dam on an opposing side of the dowel pin;
the rubber dam covered by a hydrophilic, porous window;
a load cell secured to the inner frame;
the load cell engaging the dowel pin;
the dowel pin exerting a force on the load cell; and
an outer frame secured to the inner frame.

2. The device of claim 1, wherein the outer frame slides over the inner frame and a pair of end caps close off and seal the ends of the outer frame.

3. The device of claim 1, wherein the load cell acts as a strain gauge transferring the force exerted on it as a change in electrical voltage that can be converted to a soil water tension (SWT) measurement.

4. The device of claim 1, wherein the polymer is sodium polyacrylate.

5. The device of claim 3, comprising between 0.3 and 0.4 g of sodium polyacrylate.

6. The device of claim 1, wherein
the strain gauge measures how strongly the polymer is pushing against the strain gauge;
that in turn depends on how much water the polymer has absorbed; and
how much water is available.

7. The device of claim 1, wherein
the pressure on the strain gauge can be measured with microcontrollers; and
this information can be used to determine when a crop needs to be irrigated.

8. The device of claim 1, wherein
the polymer inside the polymer chamber expands to reach equilibrium with the water outside the chamber;
that increases the pressure inside the chamber, which is transferred to a strain gauge via the dowel pin;
the strain gauge sends a voltage to a data logger and computer; and
the pressure buildup is measured via a change in voltage.

9. The device of claim 1, further comprising
a data logger; and
a computer; wherein
the strain gauge sends a voltage to the data logger and computer;
pressure buildup is measured via a change in voltage from the strain gauge; and
the measured change in voltage can be converted to soil water tension (SWT).

10. The device of claim 1, wherein the load cell is a microelectromechanical systems (MEMS) barometer.

11. The device of claim 1,
wherein the inner frame is comprised of one or more tensiometers; and
the plurality of tensiometers measure SWT at various and discrete depths in the soil profile.

12. The device of claim 11, wherein a plurality of tensiometers are spaced evenly apart across the inner frame of the device so that multiple measurements can be taken at multiple depths in the soil by the device corresponding to the spacing of the tensiometers.

13. The device of claim 11, wherein a plurality of tensiometers are spaced evenly apart across the inner frame of the device so that multiple measurements can be taken at multiple locations across a plane of soil by the device corresponding to the spacing of the tensiometers.

14. The device of claim 1, wherein one or more of the tensiometers are connected to an automated irrigation control system for use in lawns, gardens, nurseries, greenhouses, and farms.

15. A tensiometer comprising
a polymer chamber;
the polymer chamber engaging a dowel pin on one side;
the polymer chamber covered by a rubber dam on an opposing side of the dowel pin;
the rubber dam covered by a hydrophilic, porous window;
a load cell;
the load cell engaging the dowel pin;
the dowel pin exerting a force on the load cell; and
an outer frame secured to the inner frame.

16. The device of claim 15, wherein the load cell acts as a strain gauge transferring the force exerted on it as a change in electrical voltage that can be converted to a soil water tension (SWT) measurement.

17. The device of claim 16, wherein the polymer is sodium polyacrylate.

18. The device of claim 15, wherein the polymer is synthesized into macro-sized particles that cannot leak out through the rubber dam or hydrophilic, porous window.

19. The device of claim 18, wherein the polymer is synthesized into macro-sized particles >50 um in size.

20. The device of claim 15, wherein the polymer material is selected from one of the following materials:
polyethylene glycol,
sodium polyacrylate,
polyvinyl alcohol,
polyvinyl pyrolidone,
cross-linked polyethylene glycol,
cross-linked sodium polyacrylate,
cross-linked polyvinyl alcohol, and
cross-linked polyvinyl pyrolidone.

21. The device of claim 15, wherein the or hydrophilic, porous window is selected from one of the following materials:
a lightweight cork-filled PLA-based filament which is gravimetrically filled with approximately 30% cork fibres;
a 3D printer filament containing a mixture of at least 40% grinded wood particles in combination with binding polymers;
a 3D printer filament made from nuisance algae;
a 3D printer filament with a softening temperature (Vicat) of 115′ C. and high mechanical resistance;
a 3D printer filament with a silk surface; and
a 3D printer filament made from bio-polymers sourced from renewable materials like wood and other plants; and
a 3D printer filabment made from about 70% colorfabb PLA and 30% recycled woodfibers.
